(19) United States
(12) Patent Application Publication
(10) Pub. No.: US 2004/0105826 A1
(43) Pub. Date: Jun. 3, 2004

(54) Colored sunscreen compositions

(75) Inventors: David S. Soane, Piedmont, CA (US); Toshiaki Hino, Berkeley, CA (US)

Correspondence Address: JACQUELINE S LARSON, P O BOX 2426, SANTA CLARA, CA, 95055-2426, US

(73) Assignee: Cosmetica, Inc.

(21) Appl. No.: 10/721,442

(22) Filed: Nov. 24, 2003

Related U.S. Application Data
(63) Continuation of application No. PCT/US02/18277, filed on Jun. 6, 2002.
(60) Provisional application No. 60/297,155, filed on Jun. 8, 2001.

(51) Int. Cl. A61K 7/42

(52) U.S. Cl. 424/59

(57) ABSTRACT

This invention is directed to sunscreen formulations that exhibit both UV absorption and skin coloring properties. More particularly, the colored sunscreen preparations of the invention comprise a colored nanostructure that is reactive to skin or capable of being immobilized onto the skin. The colored nanostructure comprises a particulate sunblock agent in intimate relationship with a coloring agent or a colored polymeric nanomatrix. These colored sunscreen compositions provide improved retention of sunblock and coloring agents on the skin.

The present invention is a continuation of co-pending International Patent Appln. No. PCT/US02/18277, filed Jun. 6, 2002 and designating the United States of America, which application claims the benefit of Provisional U.S. patent application Ser. No. 60/297,155, filed Jun. 8, 2001; the entire disclosures of both of which are incorporated herein by reference.

### FIELD OF THE INVENTION

The present invention relates to sunblock or sunscreen compositions for screening or blocking UV and other harmful radiation, the compositions also exhibit skin coloring properties.

### BACKGROUND OF THE INVENTION

The harmful effects of solar radiation are well known. The UVB (290-320 nm) portion of the solar spectrum is largely responsible for erythema (sunburn) and cancer (M. M. Rieger, Cosme.t Toiletries, 102(3):91 (1987); C. Taylor, Skin Cancer Foundation J., 4:90 (1986)). The UVA (320-400 nm) portion of the solar spectrum, which penetrates more deeply into the skin than the UVB radiation, is believed to be responsible for skin aging and premature wrinkling by decreasing the elasticity of the skin (L. H. Kligman, F. J. Akin, and A. M. Kligman, J. Invest. Dermatol., 84:272 (1985)). Long exposure to UVA radiation also causes skin cancer such as deadly melanomas.

Sunblock (or sunscreen) formulations have evolved over the years. The active ingredients blocking broad spectrum UV (both UVA and UVB radiation) have shifted from traditional (aromatic) organic compounds (such as p-aminobenzoic acid, octyl methoxycinnamate or 2-ethylhexyl-p-methoxycinnamate, 2-ethylhexyl salicylate or octyl salicylate, oxybenzone, benzophenone, avobenzone, homosalate, etc.) to fine metal oxide particles, such as titanium dioxide, zinc oxide, silica, iron oxide, and the like. Compared to organic UV absorbing compounds, inorganic sunscreens based on metal oxides are physiologically inert and cause little skin irritation. For example, zinc oxide is classified by Food and Drug Administration as a Category I skin protectant. Without being limited by theory, it is believed that these inorganic materials provide a sunscreening benefit through reflecting, scattering, and/or absorbing harmful UV and visible radiation.

Particulate titanium dioxide (TiO₂) and zinc oxide (ZnO) are used extensively in coatings and plastics. As a UV absorber, zinc oxide reportedly has the highest ultraviolet absorption of all commercial pigments over the entire spectrum of UV radiation and is commercially available in a variety of grades and sizes, including a small particle size material (60-80 nm) that is used to make sun-blocking agents. Titanium dioxide is less effective than zinc oxide in protecting against long-wave UVA radiation.

As a reflector to block solar radiation, titanium dioxide interacts optimally with light that is a little more than twice its particle diameter, strongly reflecting this radiation. For an application at visible wavelengths, 250 nm (or 0.25 μgm) TiO₂ is commonly used. Twice this value (500 nm) is near the center of the visible region of the spectrum. Ultrafine TiO₂ is available as a UV blocker. Particles as small as 20 nm can be obtained commercially and are used in applications such as clear varnishes and sunscreens. At these sizes, the material is completely transparent in the visible range but will block UV light. Somewhat larger particles (30-35 nm) will start to give the material they are dispersed in some cloudiness because of the distribution of particle sizes in the commercial mixture. Larger particles produce a white color. For a large particle size material, the product based on titanium dioxide is more opaque than that based on zinc oxide because titanium dioxide is less transparent to the visible wavelengths of light than zinc oxide.

The most prevalent particulate sunscreen formulations contain micronized zinc oxide or titanium oxide. Various inactive ingredients are incorporated to help dispersion stability and sunscreen retention even under prolonged water immersion conditions. Surfactants, thickeners, oils, waxes, silicones, vitamins, fragrances, preservatives, antioxidants, and even herbal extracts, are commonly found in commercial sunblocks. The use of microparticles is a growing trend, as traditional UV absorbers based on organic compounds may be absorbed through the skin, causing potential systemic problems.

Formulations containing micronized metallic oxides are opaque white and can be gritty and shiny when applied to the skin. In addition, undesirable skin whitening often occurs, which is caused by non-uniform distributions of particles on the surface of the skin. Such non-uniform distributions of particles result from a poor dispersion of particles in a carrier prior to the application to skin. Particle-based formulations generally make the wearer's skin appear pale. In fact, most, if not all commercial sunscreens are either colorless or white.

Despite the known hazards of exposure to UV radiation, many people seek a tanned look by sunbathing and/or by visiting tanning salons. However, those people who wish to obtain a tanned look quickly have to decrease the use of sunscreens because the very benefits of sunscreens are to delay the tanning process by blocking UV radiation. In addition, the use of sunscreens does not completely eliminate the harmful effect of UV radiation because moderate tanning may also cause the same effect as sunburn. Consequently, getting a tanned look is always accompanied by an increased risk of skin damage.

Most commercial sunscreens perform only one dedicated function of UV blockage, and do not offer other cosmetic benefits such as artificial tan and/or masking of defects/discoloration. Artificial tanning lotions or solutions, on the other hand, generally do not possess effective UV blocking properties. Thus, there is a need for skin care products which function as sunscreens as well as artificial tanning lotions or colorants.

Skin care products are also desired to provide an effective deposition and attachment of cosmetic agents to the skin. Preferably, the attachment of cosmetic agents would be reversible such that the agents can be easily and safely removed at the discretion of wearers.

### SUMMARY OF THE INVENTION

This invention is directed to sunscreen formulations that exhibit both UV absorption and skin coloring properties. More particularly, the colored sunscreen preparations of the invention comprise a particulate sunblock agent and a coloring agent in intimate relationship with each other. These colored sunscreen compositions are reactive to skin or capable of being immobilized onto the skin, providing improved retention of sunblock and coloring agents on the skin.

The coloring agent may be chemically anchored on the sunblock particle, or it may surround or encapsulate the particle, or the sunblock particle may surround or encapsulate the coloring agent. The coloring agent may also be physically dispersed together with the sunblock particle in a cosmetic carrier. The coloring agent may, in one embodiment, be present in the colored sunscreen composition as a dye-polymer conjugate or colored polymeric nanomatrix.

The “coloring agent” is selected from pigments and dyes, including UV-absorbing dyes. In a presently preferred embodiment, the coloring agent and a polymeric nanomatrix constitute a colored polymeric nanomatrix. The “colored polymeric nanomatrix” comprises a pigment or dye in intimate relationship with a polymer to give a “dye-polymer conjugate”. The nanomatrix may further comprise particulate-reactive functional groups or other characteristics that allow it to be covalently bound to or otherwise immobilized onto or around a particulate sunblock agent. This invention describes a systematic approach where nanoscopic objects or structures comprising a dye are either shaped as a miniature sphere or particle, referred to herein as a “polymer nanosphere”, that can be attached to a particulate sunblock agent, or as an invisibly small, molecular-dimensioned net that can surround or otherwise attach to a particulate sunblock agent, referred to herein as a “nanoscopic macromolecular network” or “nanoscopic polymer network”. The nanospheres and nanoscopic networks are constructed out of polymeric materials, which can be either naturally occurring or synthetic. The natural kind can be modified or derivatized by well-established organic chemistry. The synthetic type can be specially designed to exhibit custom-tailored properties.

Regardless of the geometrical features, the nanoscopic nature of the dye-polymer conjugate entities being engineered offers several advantages: (1) color formation is performed off-line; toxic precursors and in situ chemical reactions are no longer needed; (2) the imparted color can be controllably retained or removed; (3) the coloring agents can be attached to a polymeric structure, impeding their uptake by the human body; (4) numerous colors and depths of shade can be developed based on the same general framework; and (5) the structure provides a means for depositing and attaching the coloring agents to the skin. This provides formulations that are safe and unlikely to penetrate the skin and become absorbed systemically, and that are long lasting, even in water, while having the artificial color controllably removable.

The present invention is also directed to the deposition and attachment of sunblock and coloring agents to the surface of the skin. The colored polymeric nanomatrix comprises polymers which provide a means to be immobilized on the skin, enabling an effective delivery and long-lasting benefits of cosmetic agents. Alternatively, the particulate sunblock agent may be made to be reactive to skin or capable of being immobilized onto the skin. In one embodiment of this invention, the attachment of the colored nanostructure to the skin is reversible, in which both attachment and detachment can be carried out under physiologically acceptable conditions.

Methods are provided for synthesizing a UV-protective sunscreen composition exhibiting a skin coloring property. Such methods include coupling a particulate sunblock agent to the dye or pigment molecules or the colored polymeric nanomatrices by, for example, silane coupling agents or by replacing the hydroxyl groups on the particle surface by either an ether or ester linkage. Alternatively, a particulate sunblock agent can be encapsulated by nanoscopic networks of colored polymers to provide an organic layer surrounding the particulate. The monomeric or polymeric layer may be covalently attached to the sunblock agent particle or it may be crosslinked to form a polymeric shell around the particle. The resulting dye-functionalized particulate sunblock agents are then mixed with inactive carriers and components for forming, e.g., a cream, cream gel, milk, lotion, or other composition for application to the skin. Alternatively, the coloring agent molecules or colored nanomatrices and the UV sunblock particles may be physically dispersed together in a carrier to form a cream, cream gel, milk, lotion, or other composition for application to the skin.

### DETAILED DESCRIPTION OF THE INVENTION

The terms “a” and “an” used herein and in the appended claims mean “one or more”.

The colored sunscreen composition of the present invention comprises a particulate sunblock agent and a coloring agent in intimate relationship with one another.

The term “particulate sunblock agent” as used herein refers to the solid physical sunblocks such as titanium dioxide, zinc oxide, silica, iron oxide, and the like, which provide a sunscreening or protective benefit through reflecting, scattering, and/or absorbing harmful UV and/or visible radiation. Particulate sunblock agents may be uncoated or coated with surface treatment compounds such as silica. In a presently preferred embodiment, the particulate sunblock agent is selected from titanium dioxide and zinc oxide.

In addition to the particulate sunblock agent, the composition of this invention may also contain a traditional organic sunblock agent. The term “organic sunblock agent” as used herein refers to the UV absorbing organic compounds such as p-aminobenzoic acid (PABA) and PABA esters, cinnamates such as octyl methoxycinnamate and 2-ethylhexyl-p-methoxycinnamate, salicylates such as 2-ethylhexyl salicylate and octyl salicylate, oxybenzone, benzophenone, avobenzone, homosalate, and the like. The organic sunblock agent may be physically dispersed together with the particulate sunblock agent in a cosmetic vehicle. The organic sunblock agent may also be attached to or encapsulated in a polymeric nanostructure which may contain a particulate sunblock agent and/or a coloring agent.

The term “coloring agent” as used herein and in the appended claims refers to pigments and dyes including, but not limited to, direct dyes, mordant dyes, reactive dyes, UV-absorbing dyes, photochromic dyes, fluorescent dyes, phosphorescent dyes, and optical brighteners. Both organic and inorganic coloring agents fall within the scope of this invention. The terms “coloring agent”, “colorant” and “dye” are used interchangeably herein and in the appended claims.

The term “dyestuff” as used herein refers to dye and pigment molecules or their aggregates.

A dye or pigment of particular interest for use in the present invention is melanin, a naturally occurring pigment in human hair and skin. Melanin aggregates are capable of exhibiting a range of colors, depending on their size and surface concentration. Melanin-functionalized particulate sunblock agents and/or melanin-polymer conjugates dispersed with UV blockers are a salient example of the present invention. Formulations based on melanin mimic nature closely.

The terms “nanostructure” and “nanomatrix” as used herein refer to objects characterized by a dimension of 1 nanometer to 1 micron (1 micrometer, or 1000 nanometers). Nanostructures may be organic or inorganic. In the present invention, preferred nanostructures comprise polymers. Polymeric nanostructures, as disclosed herein, may be classified into i) particulate polymeric nanomatrices or polymeric nanospheres, and ii) non-particulate polymeric nanomatrices. The terms “particulate nanomatrix” and “nanosphere” are used interchangeably. The examples of particulate polymeric nanomatrices include, but are not limited to, latices, pseudo latices, emulsion droplets, micelles, proteins, and liposomes. The examples of non-particulate polymeric nanomatrices include, but are not limited to, linear polymers, including homopolymers and copolymers, graft copolymers, comb polymers, branched polymers, star polymers, dendrimers, and lightly crosslinked polymers or nanogels.

The term “colored polymeric nanomatrix” as used herein and in the appended claims refers to colorant-containing polymeric nanostructures in which the pigments or dyes are in intimate relationship with the polymers.

The terms “colored sunblock agent” and “dye-functionalized sunblock agent” as used interchangeably herein and in the appended claims refer to particulate sunblock agents conjugated with pigments, dyes, or colored polymeric nanomatrices in intimate relationship with the sunblock agent particles.

The term “colored nanostructure” as used herein and in the appended claims refers to colorant-containing organic or inorganic nanostructures. Both “colored polymeric nanomatrix” and “colored sunblock agent” fall under the definition of “colored nanostructure”.

By “intimate relationship” is meant that the dyestuff is surrounded by, contained within, chemically attached to or otherwise in permanent or semi-permanent relationship with the nanostructure, including the polymeric nanomatrix and the particulate sunblock agent.

The term “mordant” as used herein refers to the chemicals that fix the coloring agents or colored nanostructures in or on a substance by combining with the coloring agents or colored nanostructures to form insoluble colorant-containing compounds. An example of mordant is a species that contains a metal atom with an oxidation number of 2 or higher.

The terms “payload” and “payload agent” as used herein refer collectively to any material or agent that would be desirable for permanent or semi-permanent attachment to or treatment of human skin. It may modify a property of skin or may add new and desirable properties to the skin. The payloads are also referred to herein as “pendant groups”. The payload may be, but is not limited to, dyes or coloring agents, pigments, opacifying agents, scents and fragrances, drugs and pharmaceuticals, softeners, insect repellents, antibacterials and antimicrobals, and the like. While the following discussions herein are directed to certain exemplary agents, it is important to note that other materials having any desirable activity or characteristic suitable for skin treatments may also be incorporated into polymeric nanostructures according to the teachings herein and are included within the scope of this invention.

The term “particulate-reactive functional groups” as used herein refer to functional groups that can bind or attach to particulate sunblock agents. The particulate-reactive functional groups may also be functional groups that can bind to a linker molecule that will in turn bind or attach to the sunblock particle.

The term “skin-reactive functional groups” as used herein refer to functional groups that can bind or attach to the surface of the skin. The skin-reactive functional groups may also be functional groups that can bind to a linker molecule that will in turn bind or attach to the surface of the skin.

By “change in the thermodynamic balance” is meant the change in the thermodynamic variables such as temperature, pressure, pH, ionic strength, and mixture composition which determine the phase equilibria of mixtures.

The colored sunblock agents of the invention may be obtained, in one embodiment, by forming a covalent bond between a coloring agent and a particulate sunblock agent by reacting the coloring agent with the sunblock agent. In addition, the surface of the sunblock agent may be first coated with an anchoring agent, which is then reacted with the coloring agent. A coupling agent may also be chosen for bonding further with other coloring agents and/or polymers, which may be colored or uncolored, to form a polymeric chain, network, or shell containing more than one kind of coloring agents.

Another approach to form a colored sunblock agent is to polymerize a colorant-containing monomer mixture around a particulate sunblock agent. The colorant may be reactive or non-reactive. A polymerizable surfactant may be used to improve the dispersion of particles and/or to introduce a functional group to the colored sunblock agent through the head group of the surfactant. The functional group may be chosen to provide a means for the deposition and attachment of colored sunblock agent to the skin.

The colored sunblock agents of the invention may also be formed by contacting a particulate sunblock agent with a set of colored polymeric nanomatrices. For example, polymer-encapsulated particulate sunblock agents can be obtained by forming a colored nanoscopic polymer network around a particulate sunblock agent by precipitating the colorant-containing polymers in the presence of particulate sunblock agents. In another embodiment, the colored polymeric nanomatrices comprising polymerizable groups assemble around the particles and then are polymerized, with or without crosslinking, into a polymeric network or shell surrounding and encapsulating the sunblock agent.

In the present invention, the surface of the colored polymeric nanomatrices may contain particulate-reactive functional groups for binding or attachment to the particulate sunblock agents, providing permanent or semi-permanent attachment of the dyestuff. Alternatively, the surface of the nanomatrices may include functional groups that can bind to a linker molecule that will in turn bind or attach the colored polymeric nanomatrix to the sunblock particle.

In one embodiment of the invention, the colored sunblock agents are obtained by forming a covalent bond between a particulate sunblock agent and a colored polymeric nanomatrix by reacting the colored polymeric nanomatrix with the sunblock agent. One or a mixture of colored polymeric nanomatrices may be used with the coupling agent to form a polymeric chain or network comprising colored polymeric nanomatrices. In a presently preferred embodiment, the colored polymeric nanomatrices comprise non-particulate polymeric nanomatrices. In another embodiment, the colored polymeric nanomatrices comprise polymeric nanospheres.

The attachment mechanism between a particulate sunblock agent and a colored polymeric nanomatrix is not limited to the formation of a covalent bond between them. The attachment mechanisms also include, but are not limited to, ionic attraction, van der Waals interactions, and hydrogen bonds. When a colored polymeric nanomatrix is conjugated with a particulate sunblock agent, the colored polymeric nanomatrix may surround or encapsulate the particulate sunblock agent. Conversely, the particulate sunblock agent may surround or encapsulate the colored polymeric nanomatrix.

The colored polymeric nanomatrix may be in the form of a dye-containing nanosphere. In dye-containing nanospheres, the dyestuff is entrapped, that is, surrounded by or contained within a polymer shell or matrix. The nanosphere may comprise a polymeric shell surrounding the dyestuff or it may comprise a three-dimensional polymeric network entrapping the dyestuff, both of which are referred to herein as a “polymer shell”. Similarly, the dyestuff may surround a polymer shell by reacting the dyestuff with the polymer shell. The nanospheres may be made of non-toxic, non-allergenic polymers. Many polymers have been approved by the FDA for topical usage. Silicones and cellulosics, among many others, are salient examples. Synthetic hydrocarbon-based polymeric systems are equally suitable alternatives. Proteins or synthetic peptides can also be used for this purpose.

The advantage of nanospheres is that when functional groups are present on the surface of the dye-containing nanospheres for binding or attachment to particulate sunblock agents, the chemical linkage on the surface of nanospheres does not involve the molecules of the dyestuff if the dyestuff is surrounded by or contained within a polymer shell. The pigment or dye agents are physically entrapped within the nanosphere, thus requiring no chemical modifications of the dyestuff molecules themselves. The resulting encapsulated dye preparations do not change the inherent character of the dyestuff.

The dye-containing nanospheres based on polymer lattices may be formed via one of several methods of encapsulation known in the art, such as interfacial polymerization, microemulsion polymerization, precipitation polymerization, and diffusion. Multi-component mixture preparation followed by atomization/spraying into a drying chamber is yet another processing scheme.

The dye-containing nanospheres are formed by contacting a dyestuff with a set of monomers, oligomers, or polymers (referred to herein as a “polymeric set”). The monomers, oligomers, or polymers assemble around the dyestuff and then are polymerized, with or without crosslinking, into a polymeric network or shell surrounding the dyestuff.

Alternatively, a nanosphere optionally having particulate-reactive functional groups on its surface can first be prepared by polymerizing a polymeric set, after which the dyestuff can be exposed to the bead under suitable conditions such that the dyestuff is absorbed into and entrapped in the polymeric network or shell, to provide the particulate-reactive dye-containing nanosphere.

The polymeric set in one embodiment includes at least some components that provide particulate-reactive functional groups on the surface of the final polymeric nanosphere, which will bind to sunblock particles to give the dye-functionalized particulate sunblock agents of the invention.

Particular monomers, oligomers, or polymers useful in forming the nanospheres of the present invention are those that contain amine, hydroxyl, or sulfhydryl monomers or polymers combined with amine-, hydroxyl-, or sulfhydryl-reactive monomers or polymers.

Nanospheres are but one geometry as a possible dye carrier. Dye molecules can also be attached to linear, branched, or lightly crosslinked polymer carriers to give dye-containing nanoscopic networks. For example, polymers containing free amine groups are useful to form polymeric or oligomeric dyes because amines are well known to react with a variety of dyes.

Dye molecules may also be entrapped inside the nanoscopic networks comprising particulate-reactive functional groups. The dye-containing networks are then chemically attached to sunblock particles via these functional groups. Alternatively, the networks are then crosslinked together via the appropriate reactive crosslinking functional groups to form a net or shell around a sunblock particle to give the dye-functionalized particulate sunblock agents of the invention.

One group of polymers useful as nanomatrices in the present invention are the dendrimers and other highly branched polymers. Dendrimers also have a high degree of symmetry. Dendrimers and highly branched polymers can be designed to have a large number of one or more different types of functional groups on them. Using these functional groups, dye molecules, alkyl or siloxane chains to add softness, or other molecules of interest can be attached to the dendrimer so that it becomes a compact carrier which can transport high densities of payloads via covalent bonds. These functional groups can also be transformed into the particulate-reactive functional groups. Dendrimers are also capable of encapsulating guest molecules within their cavities. Examples of commercial dendrimers include Lupasol™ (BASF), which is a highly branched polyethyleneimine having terminal amine groups; poly(amidoamine) dendrimers (Aldrich); poly(propyleneimine) dendrimers (DSM); and BOLTORN™ polyester dendrimers (PERSTORP).

Another advantage of branched polymers is that the intrinsic viscosity of a branched polymer is lower than that of a linear analog having the same molecular weight and chemical structure. Therefore, at a given solution viscosity, the branched polymers allow for the use of higher molecular weight polymers in comparison to the linear polymers of the same composition. The use of higher molecular weight polymers may increase the efficiency of deposition and retention of polymeric nanomatrices on the skin when the nanomatrices are precipitated on the skin by crosslinking.

Another group of polymers useful as nanomatrices in the present invention are lightly crosslinked polymer networks or nanogels, which are polymeric networks characterized by dimensions of 1 nanometer to 1 micron (1 micrometer, or 1000 nanometers). Nanogels exhibit the properties of cross-linked gels and colloidal particles. They can be dispersed as fine dispersions and loaded with payloads, which may be physically entrapped inside the nanogels or chemically grafted to the nanogels through pendant or terminal groups. Nanogels may be synthesized by emulsion polymerization or by crosslinking of polymers with suitable crosslinking agents using an emulsification/solvent evaporation technique. Nanogels may be formed as interpenetrating polymer networks or semi-interpenetrating polymer networks comprising more than one kind of chemically dissimilar homopolymers and/or copolymers.

Other useful polymers include, but are not limited to, amine-containing polymers or oligomers such as poly(ethylenimine), poly(allylamine hydrochloride), poly(lysine), or poly(arginine); and carboxyl-containing polymers or oligomers such as poly(acrylic acid), poly(itaconic acid), poly(maleic anhydride), a copolymer containing maleic anhydride units, a polymer with —C₆H₅COOH groups, or poly(methacrylic acid).

In one embodiment, silicones are incorporated into the colored sunscreen composition of this invention. Silicones are preferred cosmetic agents which provide a desirably shiny appearance and smooth skin and hair feel. In the present invention, silicones may be incorporated into the composition by using silicone-based or silicone-grafted nanostructures.

Nanomatrices may also be formed from the amphiphilic block copolymers which comprise hydrophobic and hydrophilic segments. Such block copolymers in aqueous solutions are known to self-assemble into core-shell type micelles in which the core and shell parts of the micelles comprise, respectively, the hydrophobic and hydrophilic segments of the block copolymer. The amphiphilic block copolymers self-assemble through, e.g., hydrophobic interaction, electrostatic interaction, and metal complex formation, which can be induced by a change in the thermodynamic balance of the medium containing the block copolymers. Examples of amphiphilic block copolymers include, but are not limited to, polyoxyethylene-co-polyoxypropylene and polyoxyethylene-co-polyaspartic acid block copolymers.

Self-assembling amphiphilic block copolymers provide several ways to form dye-functionalized particulate sunblock agents. For example, a coloring agent is first reacted with the amphiphilic block copolymer, which is then transformed into a micelle. A coloring agent and additional payloads may also be encapsulated by a micelle formed from the amphiphilic block copolymer. The dye-functionalized micelles are then attached to a particulate sunblock agent. The dye-functionalized block copolymer may also encapsulate a particulate sunblock agent by transforming into a micelle around the particulate sunblock agent or by adsorbing on the surface of the particulate sunblock agent.

When the formulation of the present invention includes block copolymers, block copolymers may also be designed to function as the dispersants for the colored nanostructures. In that event, the block copolymers are adsorbed to the colored nanostructures and improve the dispersion of nanostructures by preventing the nanostructures from flocculating. These adsorbed block copolymers may also be used to provide a means for attaching the colored nanostructures to the skin by, for example, comprising skin-reactive functional groups or crosslinkable groups which, upon crosslinking reaction, precipitate nanostructures to the skin.

Other examples and further discussions of dye-containing polymeric nanostructures are presented in International Patent Publn. No. WO 01/78663, the disclosure of which is incorporated herein by reference.

Skin Attachment Mechanisms

Well-established encapsulation techniques exist to encase the right amount of dye in particles of controlled size distribution. Well-established techniques are also available to coat the surface of particulate sunblock agents by polymers. However, in the present invention, the materials comprising the colored nanostructures (i.e., colored polymeric nanomatrices and colored sunblock agents) are chosen and/or the surface of the colored nanostructures are modified to provide an effective deposition and attachment of colored nanostructures on the surface of the skin. The colored sunscreen composition of this invention provides an improved retention of sunblock and coloring agents on the skin.

The colored nanostructures may be immobilized on the surface of the skin through the following attachment mechanisms: direct attachment, precipitation induced by a change in the thermodynamic balance of the medium, and precipitation by crosslinking. The bonds formed between the skin and the colored nanostructures and the bonds between the cross-linking agent and the crosslinkable groups of the colored nanostructures may be hydrogen bonds, ionic bonds, dative bonds, covalent bonds, or mixtures of thereof. As a direct attachment of colored nanostructures, antibodies may also be attached to the nanostructures to provide a means for binding the colored nanostructures to the skin. In a preferred embodiment, colored nanostructures are immobilized on the skin by crosslinking through the formation of ionically bonded crosslinks.

The colored nanostructures containing skin-reactive functional groups may be formed from the polymeric sets comprising the skin-reactive functional groups. The compounds comprising skin-reactive groups may also be grafted on the surface of the colored nanostructures to introduce skin-reactive groups.

The colored nanostructures comprising skin-reactive functional groups may be covalently attached to the skin through functional groups such as amines, sulfhydryls, carboxyls, and hydroxyls that are abundant in the molecules forming the skin. As examples of skin-reactive functional groups, those that are amine-reactive include, but are not limited to, isocyanates, isothiocyanates, N-hydroxysuccinimide esters, sulfonyl chlorides, aldehydes, epoxides, carbonates, anhydrides, and arylating agents. Examples of sulfhydryl-reactive groups include, but are not limited to, maleimides, disulfides, and haloacetamido compounds.

Along the backbone of the polymer constituting the nanostructures, skin-reactive functional groups may be introduced that may either be chemically reactive under mild conditions or be electrostatically interactive with complementary groups on the surface of the skin when the ionic strength or surfactant content of the medium is shifted by rinsing. Example interactions include charge-charge, dipolar, hydrogen-bonding, hydrophobic, or dehydration interactions.

The nanostructures may also be made of a polyelectrolyte with an isoelectric point in the range of alkaline pH. These nanostructures may be effectively precipitated or aggregated by using another polyelectrolyte (linear or branched polymer fixative) that possesses an acidic isoelectric point. When the skin is first exposed to the nanostructures and then is re-exposed to the second polyelectrolyte fixative, a complex forms in situ, coating the treated skin.

Another route is the use of a potent surfactant formulation to carry the colored nanostructures to the skin surface in a finely divided dispersion. Once in place, the surfactant is rinsed away, leaving the nanostructures adhering strongly to the treated skin. An example is silicone-based and silicone-grafted nanostructures such as the silicone-grafted proteins which are also conjugated with the dye. Examples of proteins include, but are not limited to, keratin, collagen, gelatin, and their derivatives. Such silicone-containing particles can be easily dispersed in a carrier using a block or graft copolymer of poly(dimethylsiloxane-co-ethylene glycol) liquid as a surfactant. The latter medium may be rinsed away by water, as the component is water-soluble, leaving the insoluble nanostructure as an adherent precipitate. In a presently preferred embodiment, silicone-grafted proteins comprise keratin, collagen, and their derivatives such as the hydrolyzed and sulfonic keratins.

Colored nanostructures may also be formed from polymers that exhibit phase separation in physiologically acceptable aqueous solutions when the thermodynamic balance of the solution is shifted by, for example, changing temperature or pH. Phase separation leads to the precipitation of polymers.

As examples of thermally induced phase separation, aqueous solutions of poly(N-isopropyl acrylamide), polyethylene glycol (PEG), polypropylene glycol (PPG), PEG-co-PPG copolymers, hydroxypropyl cellulose, methyl cellulose, and hydroxypropylmethyl cellulose exhibit phase separation upon heating, which is referred to Lower Critical Solution Temperature (LCST) behavior. N-isopropyl acrylamide is also copolymerized with the monomers comprising ionizable groups to give the copolymers exhibiting LCST behavior, which depends on the pH and ionic strength of the solution. In aqueous solutions of PEG, LCST depends on the ionic strength of the solution.

On the other hand, aqueous solutions of copolymers comprising N-acetyl acrylamide and acrylamide are known to exhibit Upper Critical Solution Temperature (UCST) behavior in which the solubility of polymers increases as the temperature rises. The LCSTs and UCSTs observed in these systems are reversible. Thus, the nanostructures formed from the above-mentioned LCST and UCST polymers may be reversibly attached to the skin by applying to the skin treated with the nanostructures a solution in which the temperature, pH, and/or ionic strength of the solution is adjusted.

As another example of reversible attachment of the colored nanostructures by precipitation induced by a change in the thermodynamic balance, colored nanostructures formed from a water-insoluble polymer are dispersed in an aqueous solution containing a cosolvent for the pair of water and the polymer comprising the nanostructures. Preferably, the cosolvent is at least partially water soluble and more volatile than water. When the skin is treated with the preparation obtained in this manner, the colored nanostructures will precipitate as the volatile cosolvent evaporates, coating the treated skin. The deposited colored nanostructures exhibit water resistance but are readily removable from the skin by washing with a solution containing the cosolvent.

When the surface of soluble colored nanostructures contain reactive functional groups for crosslinking, the nanostructures may be crosslinked by the addition of fixatives, which function as the crosslinking agents, after the skin care compositions comprising the colored nanostructures are applied to the skin. Using volatile blocking agents for the crosslinking reaction, compositions are also formulated which contain both crosslinkable nanostructures and crosslinking agents. After such compositions are applied to the skin, the blocking agents are removed by evaporation, initiating the reaction to crosslink the colored nanostructures.

As an example of precipitation of colored nanostructures by crosslinking, the nanostructures may be attached to the skin via a mordant or cationic fixing agent. Carboxyl-, phosphate-, phosphonate-, sulfate-, and sulfonate-containing polymers can be complexed with alkaline earth metal that have very low toxicity, such as Mg²⁺, Ca²⁺, and Sr²⁺, which form crosslinks between the above-mentioned functional groups. Thus, for example, a soluble polymer that contains, for example, carboxyl groups and one or more payloads, such as dye molecules or compounds that add softness, is applied to the skin. In a next step, a soluble calcium or magnesium salt is added to the skin to precipitate the payload-containing polymer on the skin.

As another example of attachment to the skin via a mordant, colored nanostructures containing mordant dyes are precipitated by the mordants that are used to fix the dyes. Because mordant dyes contain crosslinkable functional groups such as carboxyl groups, colored nanostructures containing mordant dyes may also be complexed with alkaline earth metal such as Mg²⁺, Ca²⁺, and Sr²⁺, which will form crosslinks between the functional groups of mordant dyes.

Functionalized siloxanes can further refine the precipitation principle assisted by surfactants by utilizing complexation as well. For example, siloxanes with carboxylate side groups may be precipitated by the dual use of removing the surfactant and adding a polyamine (such as polyethyleneimine in the aqueous rinse solution). Conversely, amino-substituted siloxanes can form an in situ crosslinked network with the colored nanostructures embedded within by the addition of polyacids (such as polyacrylic acid or polymaleic acid or copolymers thereof).

Complexation can also be induced by addition of polyvalent cations or anions, each reactive towards the complementary-charged surface groups. Acid-base neutralization is another example of complexation-induced precipitation/anchoring of nanostructures.

The principle of thermodynamics-induced and complexation-induced precipitation/anchoring on skin surfaces can be equally applied to other synthetic or naturally occurring nanostructures. For example, the colorant, particulate sunblock agent, and/or payload can first be chemically coupled onto a protein carrier. Numerous approaches are known to chemically modify proteins through functional groups such as sulfhydryl, amine, and carboxyl groups. This protein-payload complex is dispersed in a medium, which is then applied to the skin. A change in the thermodynamic balance of the medium, such as the change in pH and ionic strength, causes deposition of the complex on the surface of skin. Proteins can also be precipitated by the addition of non-ionic polymers or metal ions. The skin is thus treated. Since coupling is carried out chemically outside the presence of skin, traditional chemical means can be used without fear of hair degradation or skin sensitivity. Protein deposition can then be effected by simpler, milder fixative reactions.

We reiterate the power of delegating different engineering requirements to different parts of the system. The color comes from the dyestuff contained within the colored nanostructures. Yet, the controlled degree of permanency stems from the skin-attachment methodology. The above precipitation/complexation approach can be made difficult to reverse or it can be easily reversible. Reversibility can be engineered to occur only in the presence of certain specific agents. Therefore, normal skin wash or soap does not cause fading of the color. For example, functionalized silicones are difficult to wash away, unless specific siloxane-containing surfactants such as block or graft copolymers of siloxane-polyethylene glycol are used. Equivalently, complex or precipitate dissolution may or may not occur under similarly engineered rinsing conditions. Thus, the artificially-created skin color can be either preserved in a prolonged manner or reversed when desired.

Note that derivatized cellulosics can be made to function in a similar way. Synthetic polypeptides can also be used for dyestuff encapsulation. Such cellulosic or proteinaceous surfaces can be modified to exhibit varying isoelectric points, which can be exploited to tailor their precipitation/coagulation/complexation properties.

In the present invention, the colored polymeric nanomatrices may also be physically dispersed together with the particulate sunblock agents in a carrier without linking to or encapsulating the sunblock particles. In that event, effective deposition and retention of particulate sunblock agents on the skin is still possible because the sunblock agents may be physically entrapped by the network of colored polymeric nanomatrices formed on the surface of the skin.

When the particulate sunblock agents are not conjugated with the colored polymeric nanomatrices, the particulate sunblock agents are optionally conjugated with uncolored polymeric nanomatrices and/or the surface of sunblock agents is treated to provide a means for the deposition and attachment of uncolored sunblock agents to the surface of the skin. In that event, the same mechanisms may be employed as those used to fix the colored sunblock agents to the skin. In one embodiment, the colored polymeric nanomatrix is formed from a dye-conjugated protein which is further grafted with silicone. Similarly, the particulate sunblock agent is conjugated with a protein which is further grafted with silicone. These protein-based colorant and sunblock agent, which are also grafted with silicone, are then physically dispersed together in a carrier which contains a block or graft copolymer of poly(dimethylsiloxane-ethylene glycol) liquid as a surfactant.

In certain instances, the colored nanostructures also contain payloads other than the dyestuff. For example, organic UV absorbers may be included in the colored nanostructures. Payloads may also be incorporated in separate optional nanostructures which do not contain the dyestuff. The materials comprising these optional nanostructures may be chosen and/or the surface of optional nanostructures is treated to provide an effective deposition and attachment of nanostructures on the surface of the skin.

When the payload is a fragrance or a pharmaceutical agent, it is desirable for the payload to be controllably released from the nanostructure on or into the skin. Nanoparticles or nanogels can be designed so that the payload agent is embedded or entrapped within the polymeric shell or matrix of the nanoparticle or nanogel but is also able to be released from the nanoparticle or nanogel in a prolonged or otherwise controllable fashion. The release profile is programmed via the chemistry of the polymer network of the nanoparticle. The nanoparticle can be formulated with an almost infinite degree of designed characteristics via structural features, such as crosslinking density, hydrophilic-hydrophobic balance of the copolymer repeat units, and the stiffness/elasticity of the polymer network (for example, the glass transition temperature). In addition, erodible nanoparticles or other nanostructures can be developed to controllably release the payload.

Furthermore, the polymer matrix may contain components that react or respond to environmental stimuli to cause increased/decreased content release. “Smart polymers” are polymers that can be induced to undergo a distinct thermodynamic transition by the adjustment of any of a number of environmental parameters (e.g., pH, temperature, ionic strength, co-solvent composition, pressure, electric field, etc.). For example, smart polymers based on the LCST transition may cut off release when exposed to warm or to hot water during washing. When cooled back to room temperature, sustained release resumes. Conversely, smart polymers based on the UCST transition may turn on release when the surface temperature of the skin rises.

Smart polymers may be formed from, but are not limited to, N-isopropyl acrylamide, acrylamide, N-acetyl acrylamide, N-acetyl methacrylamide, functionalized polyethylene glycol and polypropylene glycol, methyl methacrylate, hydroxyethylmethacrylate, octyl/decyl acrylate, acrylated urethane oligomers, vinylsilicones, and silicone acrylate.

Smart polymers may also be selected from, but are not limited to, polyvinylmethyl ether, polyvinylethyl ether, polyvinyl alcohol, polyvinyl acetate, polyvinyl pyrrolidone, polyhydroxypropyl acrylate, cellulose, methyl cellulose, hydroxyethyl cellulose, hydroxypropyl methyl cellulose, hydroxypropyl cellulose, ethyl hydroxyethyl cellulose, hydrophobically-modified cellulose, dextran, hydrophobically-modified dextran, agarose, low-gelling-temperature agarose, and copolymers thereof.

If crosslinking is desired between the polymers, multifunctional compounds such as bis-acrylamide and ethoxylated trimethylol propane triacrylate and sulfonated styrene may be employed. In presently preferred embodiments, the smart polymers comprise polyacrylamides, substituted polyacrylamides, copolymers based on polyethylene glycol, polyvinylmethyl ethers, and modified celluloses.

The polymeric set can be chosen to give either hydrophobic or oleophilic nanoparticles, allowing a wider array of bioactive compounds or drugs to be comfortably entrapped within. Where the particles are hydrophilic, they are easily dispersible in a stable aqueous suspension or emulsion by surfactants, which can subsequently be washed away without affecting the performance of the payload agent within. The inherent thermodynamic compatibility of the agents and the polymeric shell or matrix material can increase the loading level per particle.

The sunscreen formulations of the present invention are prepared by mixing the colored sunblock agents with cosmetically or dermatologically acceptable carriers and components for forming, e.g., a cream or lotion by methods well-known in the art. Alternatively, the colored polymeric nanomatrices are dispersed with the particulate sunblock agents in the carrier and other cream or lotion components. This and all other formulations and solutions of the invention may additionally contain fragrances, deodorants, wetting agents, additional UV blockers, oxidizing agents, antioxidants, opacifiers, thickeners, film forming polymers, reducing agents, defoamers, pigment dispersants, surfactants (anionic, cationic, nonionic, amphoteric, zwitterionic, or mixtures thereof), sequestering agents, medicines (drugs), dispersing agents, conditioners, limited quantities of organic solvents, antibacterial agents, preserving agents, and the like, as well as mixtures thereof.

The colored nature of the sunscreen formulations of the invention allows their use, in addition to their sunblocking protection, as indicators of when that protection is no longer present. That is, as the lotion or cream is washed or worn away, the dye-containing particles will also necessarily wash away, resulting in a fading or disappearance of the color. The wearer then knows to reapply the sunblock.

The following examples are intended to illustrate some, but not all, of the concepts described in this disclosure, and are in no way intended to limit it. One skilled in the art would also see that different ideas from different examples or from the above explanation could be combined to yield other possible ways of treating skin.

### EXAMPLES

### Example 1

One or more of the same or different dye molecules are covalently bonded, by methods known in the art, to an amine-containing polymer or oligomer such as poly(ethylenimine), poly(allylamine hydrochloride), or poly(lysine). (An oligomer or polymer of arginine would be expected to behave similarly.) The dye-conjugated polymer is then precipitated on a particulate sunblock agent to give the colored sunblock agent.

Skin is wet with a solution containing this colored sunblock agent. In some cases it may be necessary to rinse away excess material. To set or cure the polymer-coated sunblock agent, the skin is then exposed to a polymer that contains carboxyl, sulfate, sulfonate, phosphate, or phosphonate moieties. Examples of such polymers include DNA, poly(acrylic acid), poly(itaconic acid), poly(maleic anhydride), copolymers containing maleic anhydride units, a polymer with —C₆H₅COOH groups, poly(methacrylic acid), or poly(styrene sulfonate, sodium salt). Excess material is then rinsed away. An electrostatic interaction holds the two polymers together, greatly decreasing the solubility of the complex.

### Example 2

One or more dye molecules are covalently bonded to a carboxyl-containing polymer or oligomer such as poly(acrylic acid), poly(itaconic acid), poly(maleic anhydride), a copolymer containing maleic anhydride units, a polymer with —C₆H₅COOH groups, or poly(methacrylic acid). This polymer is then precipitated on a particulate sunblock agent to give the colored sunblock agent. Skin is wet with a solution containing this colored sunblock agent. Excess material is then rinsed away. To set this polymer-coated sunblock agent, the skin is exposed to a polycation (polymer or oligomer), such as poly(ethylenimine), poly(allylamine hydrochloride), poly(lysine), poly(arginine), or poly(diallyldimethylammonium chloride).

### Example 3

Skin is exposed to a solution containing one or more polymeric or oligomeric dyes, as described in Example 1 (polycations), and particulate sunblock agents. It may be necessary to rinse the skin after this first treatment. The skin is then exposed to a solution that contains one or more polymeric or oligomeric dyes (polyanions), as described in Example 2, and it is rinsed. The sunblock agent is trapped in the complex formed between two polymers and precipitated on the skin.

### Example 4

One or more dye molecules is covalently attached to a polymer or oligomer of ethylenimine such as triethylenetetramine. In addition to an ethylenimine, any polymer with free amine groups may be used, including poly(allylamine hydrochloride) and poly(lysine). After introduction of the dye to the polymer, a group capable of chelating a metal is introduced to the colored polymer by the method disclosed by International Patent Publn. No. WO 01/78663, leaving a metal-chelating polymeric dye. This colored polymer may further be conjugated with a particulate sunblock agent. The colored polymer may also be dispersed together with a particulate sunblock agent in a carrier to give a colored sunblock composition.

The composition obtained in this manner is applied to the skin, coating the skin with the colored polymer-conjugated sunblock agent. A mordant is then applied to the skin, immobilizing the colored sunblock agent.

The deposition process discussed in this example may be reversed by the extraction of the metal atoms from the deposited polymer with, for example, ethylenediaminetetraacetic acid (EDTA) or nitrilotriacetic acid (NTA), which reverses the initial precipitation process.

### Example 5

This example makes use of silica-treated particulate sunblock agents. A silica-treated particulate sunblock agent is coupled with, for example, silane coupling agents, or the hydroxyl groups on the particle surface are replaced by either an ether or ester linkage. A dye-conjugated amine-containing polymer or oligomer is then coupled with the sunblock agent. This colored sunblock agent is then deposited on the skin by the method described in Example 1.

### Example 6

In this example, a colored polymeric nanomatrix is formed by including alkyl and/or siloxane chains which introduce softness and silkiness to the skin and hair.

An alkyl chain, which is defined herein as a linear or branched molecule that contains primarily C, CH, CH₂, and CH₃ units, is tethered to an amine-containing polymer or oligomer, such as poly(ethylenimine), poly(allylamine hydrochloride), or poly(lysine). One or more of the same or different dye molecules are also added to the polymer. Linear or branched siloxane chains may also be added to the amine-containing polymer or oligomer. This polymer is then conjugated with a particulate sunblock agent by the methods known in the art.

Skin is exposed to this polymer-conjugated sunblock agent and excess reagent may be rinsed away. The skin is then exposed to a polyanion, which may have alkyl chains, siloxane chains, or dyes tethered to it. One possible polyanion, which may act as a softener, is a copolymer of maleic anhydride and a vinyl ether of the form: CH₂═CHO(CH₂)ₙCH₃, where n is at least 2, and is preferably greater than 4.

### Example 7

A colored polyelectrolyte containing pendant groups, which modify a property of skin or which add new and desirable properties, is conjugated with a particulate sunblock agent and is deposited on the skin. An oppositely charged polyelectrolyte, which also may contain one or more pendant groups that modify a property of skin or that add a desirable property to skin, is added to the skin, condensing with the first polymer to immobilize it.

### Example 8

A colored polymer or oligomer that contains one or more pendant groups, which modify one or more properties of skin or which add one ore more desirable properties, is conjugated with a particulate sunblock agent and is deposited on the skin. Excess reagent may be washed away from the skin. A mordant is added to the deposited colored polymer-conjugated sunblock agent, immobilizing the colored sunblock agent.

### Example 9

A mordant is deposited on the skin. Excess reagent may be washed away from the skin. A particulate sunblock agent is conjugated with a colored polymer or oligomer that contains one or more pendant groups which modify one or more properties of skin or which adds one or more desirable properties. This colored polymer-conjugated sunblock agent is deposited on the skin. The mordant complexes with the colored polymer-conjugated sunblock agent to immobilize the sunblock agent.

### Example 10

A variety of molecules that impart desirable properties to skin or to the formulation can be incorporated into reactive monomers, such as in the reaction between an amine and an acid chloride. Later it will be possible to polymerize such monomers into polymers that have desirable properties, where the level or concentration of certain groups is carefully controlled.

### Example 11

To create functionalized polymers with tailored properties, a variety of molecules that add desirable properties to a polymer are added to polymers such as to poly(acryloyl chloride) and poly(acrylic anhydride), which act as scaffolds. International Patent Publn. No. WO 01/78663 discloses the reaction scheme involving these polymers.

### Example 12

N-isopropylacrylamide (NIPA) will make a polymer thermally sensitive, exhibiting LCST behavior in aqueous solutions. In other words, at low temperatures, a polymer that has NIPA (or an analogous monomer) will have a higher water solubility than at higher temperatures. Thus, a polymer can be designed that precipitates when the skin is washed with warm or hot water.

### Example 13

Thermally sensitive polymers exhibiting UCST behavior are formed by copolymerizing N-acetyl acrylamide with acrylamide. At high temperatures, these polymers will have a higher water solubility than at lower temperatures.

Colored polymeric nanomatrices are formed by reacting the dye molecules with the polymers which exhibit UCST behavior in aqueous solutions. As a payload, deodorant and/or fragrance is encapsulated or absorbed by the colored nanostructures. As the surface temperature of the skin rises and the wearer starts to perspire, the nanostructures based on UCST polymers will turn on the release of payloads encapsulated therein.

### Example 14

In this example, a set of molecules, which may be dyes, fragrances, softeners, medicines (drugs), monomers, or other molecules which modify a property of skin or which add new and desirable properties, is emulsified with a polymerizable surfactant in the presence of particulate sunblock agents. The resulting micelles encapsulate sunblock agents and are then polymerized into nanoparticles, which can be applied to the skin and then, depending on the head group of the surfactant, set with a mordant or a polyelectrolyte with a charge opposite that of the surfactant's head groups. The head groups may be designed to be analogs of EDTA or NTA so that the surfactant will be particularly effective in chelating a metal ion. Examples of polymerizable surfactants are disclosed by International Patent Publn. No. WO 01/78663.

### Example 15

A set of one or more surfactants is used to bring one or more insoluble or quite insoluble species, including polymers and oligomers, into aqueous solution. This material is applied to the skin. Upon rinsing away the surfactants in the material, the insoluble or nearly insoluble species are deposited onto skin.

As an example for the use of surfactant, a colored nanomatrix is first created, a siloxane chain is then grafted to the colored nanomatrix, and a chelating group is introduced. Preferred nanomatrices are proteins such as keratin, collagen, gelatin, and their derivatives. Siloxane and alkyl chains are expected to provide a desirably shiny appearance and smooth skin and hair feel, but another important feature of these long chains is to reduce the solubility of the polymeric or oligomeric dye.

Next, a particulate sunblock agent is conjugated with a siloxane-grafted protein and a chelating group is introduced. The particulate sunblock agent is then physically dispersed together with the colored nanomatrix comprising a siloxane-grafted protein in a cosmetic carrier containing a surfactant. Silicone-containing nanostructures can be easily dispersed in a cosmetic carrier using a block or graft copolymer of poly(dimethylsiloxane-ethylene glycol) liquid as a surfactant.

When such surfactants in the formulation are removed by rinsing, the siloxane-grafted nanomatrices may be deposited onto the skin. Addition of metal would act to increase the durability of the nanomatrices. As was the case in Example 4, the process of adding a metal is reversible using EDTA and NTA.

Siloxane chain may be grafted on an amine-containing colored polymeric nanomatrix by reacting the polymer with the siloxane chain functionalized with an epoxide group. While epoxide chemistry is a preferred embodiment of the ideas in this example, other possible reactive groups that could be used to introduce siloxane groups by means known in the art include, but are not limited to, anhydrides, acid chlorides, carboxylic acids, sulfonyl chlorides (to make sulfonamides), etc.

### Example 16

A known dye molecule, including, but not limited to acid dyes, direct dyes, reactive dyes, mordant dyes, sulfur dyes, and vat dyes, is reacted with a polymer and the polymer is deposited on the skin by one of the methods described in this document.

### Example 17

A mordant dye is coupled to a polymer or oligomer and this material is deposited on the skin. Addition of a mordant causes crosslinking of the polymer molecules through the mordant dye pendant groups.

### Example 18

A protein, which acts as a scaffold, is derivatized with dye molecules, softeners, a polyelectrolyte oligomer chain, carboxymethyl groups or other species that may impart a desirable property to the skin. The resulting protein complex is then precipitated onto skin and immobilized to one degree or another with the methods described herein, e.g., a polyelectrolyte or a mordant.

### Example 19

In the present invention, one group of polymers useful as nanomatrices are the dendrimers and other highly branched polymers. Dendrimers and highly branched polymers can be designed to have a large number of one or more different types of functional groups, such as amine groups, on them. These functional groups provide a means for conjugating with particulate sunblock agents, dye molecules, alkyl or siloxane chains to add softness, or other molecules of interest.

Dendrimers are also capable of retaining guest molecules within their cavities. For example, dyes and dendrimers are dispersed in a solvent where the dendrimers absorb the dye molecules. The solution is then precipitated into a non-solvent for the dendrimers to recover the dye-encapsulated dendrimers which are then conjugated with the particulate sunblock agents to give the colored sunblock agents.

The functional groups of dendrimers may also be transformed into skin-reactive functional groups.

### Example 20

This example makes use of an amphiphilic block copolymer as a dispersant for the colored sunblock agent and also provides a means for precipitating on the skin.

The colored sunblock agent is formed by reacting the coloring agent with the sunblock agent to attach the colorant to the sunblock agent. The surface of the sunblock agent may be first coated with an anchoring agent, which is then reacted with the coloring agent.

As a dispersant for the colored sunblock agent, an amphiphilic AB-type block copolymer is formed comprising the hydrophobic A block, which adsorbs to the colored sunblock agent, and the hydrophilic B block, which contains crosslinkable groups such as carboxyls.

The colored sunscreen composition is prepared by dispersing in the carrier the colored sunblock agent and, as a sunblock dispersant, the AB type amphiphilic block copolymer comprising carboxyl groups. This composition is applied to the skin. In a next step, a soluble calcium or magnesium salt or a polycation is applied to the treated skin to precipitate the polymer-adsorbed colored sunblock agent on the skin.

What is claimed is:

1. A colored sunscreen composition exhibiting both UV absorption and skin coloring properties, the colored sunscreen composition comprising a colored nanostructure, the colored nanostructure comprising a particulate sunblock agent in intimate relationship with a coloring agent or a colored polymeric nanomatrix and being reactive to skin or capable of being immobilized onto the skin.

2. A colored nanostructure comprising a particulate sunblock agent chemically attached to a coloring agent, and wherein the colored nanostructure is reactive to skin or capable of being immobilized onto the skin.

3. A colored nanostructure comprising a particulate sunblock agent in intimate relationship with a colored polymeric nanomatrix, wherein the colored polymeric nanomatrix comprises a coloring agent chemically attached to a polymeric nanomatrix, and wherein the colored nanostructure is reactive to skin or capable of being immobilized onto the skin.

4. A colored nanostructure according to claim 3 wherein the particulate sunblock agent is chemically attached to the colored polymeric nanomatrix.

5. A colored nanostructure according to claim 3 wherein the colored polymeric nanomatrix comprises a particulate polymeric nanomatrix.

6. A colored nanostructure according to claim 5 wherein the particulate polymeric nanomatrix is a protein or a protein derivative.

7. A colored nanostructure according to claim 6 wherein the protein or protein derivative is further grafted with silicone.

8. A colored nanostructure according to claim 3 wherein the colored polymeric nanomatrix comprises a non-particulate polymeric nanomatrix.

9. A colored nanostructure according to claim 8 wherein the non-particulate polymeric nanomatrix is selected from the group consisting of a linear polymer, a graft copolymer, a comb polymer, a branched polymer, a highly branched polymer, a star polymer, a dendrimer, and a lightly crosslinked polymer network.

10. A colored nanostructure according to claim 3 wherein the colored polymeric nanomatrix comprises silicone.

11. A colored nanostructure according to claim 3 wherein the colored polymeric nanomatrix comprises amphiphilic block copolymers.

12. A colored nanostructure according to claim 3 wherein the colored polymeric nanomatrix is in the form of a nanoscopic polymer network.

13. A colored nanostructure according to claim 3 wherein the colored polymeric nanomatrix is in the form of a polymer nanosphere.

14. A colored nanostructure according to claim 3 wherein the coloring agent comprises melanin.

15. A colored nanostructure according to claim 3 wherein the composition further comprises an organic UV absorber chemically attached to the particulate sunblock agent or to the colored polymeric nanomatrix.

16. A colored nanostructure according to claim 3 which comprises skin-reactive functional groups.

17. A colored nanostructure according to claim 3 which comprises a polymer exhibiting UCST or LCST behavior in a physiologically acceptable aqueous solution.

18. A colored nanostructure according to claim 3 which comprises functional groups that will react with a mordant.

19. A colored nanostructure according to claim 3 which comprises functional groups that will react with a cationic fixing agent.

20. A colored nanostructure according to claim 3 which comprises functional groups that will react with an anionic fixing agent.

21. A colored nanostructure according to claim 3 which comprises functional groups that will react with a fixing agent that relies on hydrophobic interactions or on hydrogen bonding.

22. A method of treating skin to provide improved retention of sunblock and coloring agents on the skin, the method comprising:
applying a colored sunscreen composition to the skin under a first set of conditions, the colored sunscreen composition comprising a colored nanostructure, the colored nanostructure comprising a particulate sunblock agent in intimate relationship with a coloring agent or a colored polymeric nanomatrix and being reactive to skin or capable of being immobilized onto the skin; and
changing the conditions to a second set of conditions such that the colored nanostructure is attached to or immobilized onto the skin.
